(19) United States
(12) Patent Application Publication
(10) Pub. No.: US 2026/0060697 A1
(43) Pub. Date: Mar. 5, 2026

(54) DUAL LUMEN CATHETER FOR VASCULAR INTERVENTION

(71) Applicant: Amit Narendra Sanghvi, Fort Wayne, IN (US)

(72) Inventor: Amit Narendra Sanghvi, Fort Wayne, IN (US)

(21) Appl. No.: 18/822,210

(22) Filed: Sep. 1, 2024

(51) Int. Cl. A61B 17/22; A61M 25/00

(52) U.S. Cl. CPC A61B 17/22; A61M 25/0026; A61M 25/005; A61B 2017/22038; A61B 2017/22079

(57) ABSTRACT

A catheter includes a plurality of lumens having at least a first lumen connected to a suction means for aspiration and a second lumen that allows passage of a guiding means, wherein the second lumen extends up to a distal transition point of the catheter. Using the second lumen for passage of the guiding means allows for unimpeded aspiration of blockages/clots through the first lumen, such as in vascular interventions.

### TECHNICAL FIELD

The present disclosure relates generally to catheters. Particularly, the present disclosure relates to multi-lumen catheters for vascular intervention.

### BACKGROUND

Existing catheters used for vascular interventions, such as embolectomy or thrombectomy, include a single lumen. Such catheters are inserted into blood vessels up to the site of a blockage, such as those caused by a blood clot. Typically, such catheters are inserted into blood vessels, and guided towards the clot using microcatheters or guide wires that are extended along the length of the lumen. The catheter is navigated/steered through the vascular network until the site of the clot is reached. Upon reaching the clot, a distal tip of the catheter is caused to come into contact with the clot. Further, the clot is aspirated towards the lumen using a suction means such that the clot is secured to the distal tip of the catheter. The catheter is withdrawn to remove the clot from the vascular network, thereby restoring circulation.

In some cases, microcatheters are also used to stab the clot such that the clot is secured to or near the distal tip of the catheters. Further, stent retrievers are also delivered to the site of the clot through the lumen. As the stent retriever is caused to expand, the mesh-like structure of the stent penetrates and engulfs the clot, thereby also securing the clot. However, the microcatheters and the stent retrievers are also likely to cause the clot to be pushed away from the distal tip of the catheter. Attempting to stab the clot also increases the chances of the clot breaking into multiple pieces (often uncontrollably), which may be undesirable in many situations.

Furthermore, microcatheters, guidewires, and/or stent retrievers occupy significant cross-sectional area of the lumen, which impedes the force of aspiration. The reduced force prevents the clot from being securely adhered to the distal tip of the catheter, and risks the clot from being separated from the distal tip when the catheter is being withdrawn. Although removing the guide wires or microcatheters may prevent the aspiration from being impeded, they may make the catheter unstable, and thereby unreliable, during aspiration.

Thus, there is a need for a catheter for vascular interventions that allow for efficient and unimpeded aspiration through the catheter. Further, there is a need for a means to secure the position of the catheter during aspiration.

### SUMMARY

In order to obviate the shortcomings of the background art, a catheter with one or more lumen is provided and illustrated in the instant disclosure.

Aspects of the present disclosure are directed to a catheter. The catheter includes a plurality of lumens having at least a first lumen connected to a suction means for aspiration, and a second lumen that allows passage of a guiding means. The second lumen extends up to a distal transition point of the catheter.

In some embodiments, the second lumen may include an orifice configured to allow the guiding means to exit the catheter.

In some embodiments, the guiding means may be configured to abut against at least one of: an inner wall of a blood vessel, or a branch vessel, for securing position of the catheter.

In some embodiments, the guiding means may be extendible either collinearly or obliquely out from the orifice with respect to the catheter.

In some embodiments, the guiding means may be at least one of: a guide wire, a microcatheter, or a stent retriever.

In some embodiments, the plurality of lumens may be enclosed in a braided sheath of the catheter.

In some embodiments, the first lumen may include a distal portion that may be flexible.

In some embodiments, the distal portion may be configured to enclose over a clot during aspiration.

In some embodiments, the catheter may include one or more transition points that divide the catheter into one or more portions, and wherein stiffness of each portion increases from a proximal end to a distal end of the catheter.

In some embodiments, the one or more transition points may include a proximal transition point and the distal transition point that divide the catheter into a proximal portion, a middle portion, and a distal portion.

In some embodiments, the proximal portion, the middle portion, and the distal portion may be about 0.2, 0.3 and 0.5 times the length of the catheter, respectively.

In some embodiments, the second lumen may include an entry opening located between a proximal end of the catheter and the distal transition point.

In some embodiments, the distal portion has a length between 0.05 times to 0.5 times that of the catheter.

In some embodiments, the first lumen has a between 0.6 to 0.8 times that of the catheter.

In some embodiments, the diameter of the first lumen increases from the distal transition point to a distal tip of the first lumen.

In some embodiments, the second lumen may have a diameter between 0.2 to 0.4 times that of the catheter.

Aspects of the present disclosure are also directed to a method for vascular interventions. The method includes inserting a catheter into a blood vessel having a clot, where the catheter may include a first lumen and a second lumen. The method further includes extending a guiding means through the second lumen to abut against a surface to secure the catheter from moving during aspiration, aspirating the clot through the first lumen using a suction means, and withdrawing the catheter to remove the clot from the blood vessel.

In some embodiments, for inserting the catheter the method may include guiding the catheter towards the clot using the guiding means.

In some embodiments, a distal tip of the first lumen encloses over at least a portion of the clot during aspiration.

In some embodiments, the surface may be at least one of: an inner wall of a blood vessel, or a branch vessel.

The following description is illustrative in nature and is not intended to be in any way limiting. In addition to the aforementioned illustrative aspects, embodiments, and features of the present disclosure, further aspects, embodiments and features will become apparent by reference to the following detailed description.

### BRIEF DESCRIPTION OF THE ACCOMPANYING DRAWINGS

The accompanying drawings illustrate the best mode for carrying out the disclosure as instantly contemplated and set forth hereinafter. The instant disclosure may be more clearly understood from a consideration of the following detailed description of the preferred embodiments taken in conjunction with the accompanying drawings where all figures are diagrammatic and not to scale. Further, like reference letters and numerals indicate the corresponding parts in various figures in the accompanying drawings.

FIG. 1A illustrates an example representation of a catheter having one or more lumens, according to embodiments of the present disclosure.

FIGS. 1B to 1D illustrate example cross-sectional views of the catheter at different lengths, according to embodiments of the present disclosure.

FIGS. 2A to 2D illustrate example representations of the catheter inserted into a blood vessel, according to embodiments of the present disclosure.

FIG. 3 illustrates a flowchart of an example method for using the catheter, according to embodiments of the present disclosure.

### DETAILED DESCRIPTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the scope of the invention as set forth.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

Reference throughout this specification to “some embodiments” or “other embodiments” or “one embodiment” or “an embodiment” or “an instance” or “one instance” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, “substantially” means largely or considerably, but not necessarily wholly, or sufficiently to work for the intended purpose. The term “substantially” thus allows for minor, insignificant variations from an absolute or perfect state, dimension, measurement, result, or the like as would be expected by a person of ordinary skill in the art, but that do not appreciably affect overall performance.

As used herein, “about” means approximately or nearly, and in the context of a numerical value or range set forth means ±15% of the numeric value.

Throughout the specification, ‘proximal’ or variations thereof may mean and include an end, a side, or a direction towards a vascular incision or a surgeon/physician inserting a catheter, and away from a blockage/clot.

Throughout the specification, ‘distal’ or variations thereof may mean and include an end, a side, or a direction away from the vascular incision or surgeon/physician, and towards the blockage/clot.

As stated, existing catheters used for vascular interventions have drawbacks, such as guiding means (like microcatheters, stent retrievers, and/or guide wires) impeding aspiration in a lumen of the exiting catheters impeding aspiration of clots/blockages, breaking or pushing the clot/blockage further away from the catheter, and the like. The present disclosure addresses at least the aforementioned problems by providing a catheter with one or more lumens, where at least one of the lumens is dedicated for aspiration, and other catheters are used for inserting the guiding means. Further, the present disclosure also provides a method for using catheters with multiple lumens, involving aspirating a first lumen while securing the catheter to a branch blood vessel using the guiding means inserted through a second lumen.

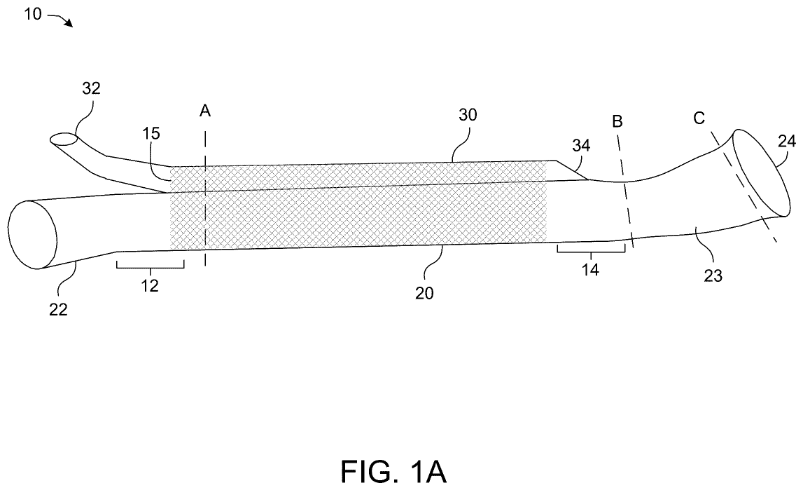

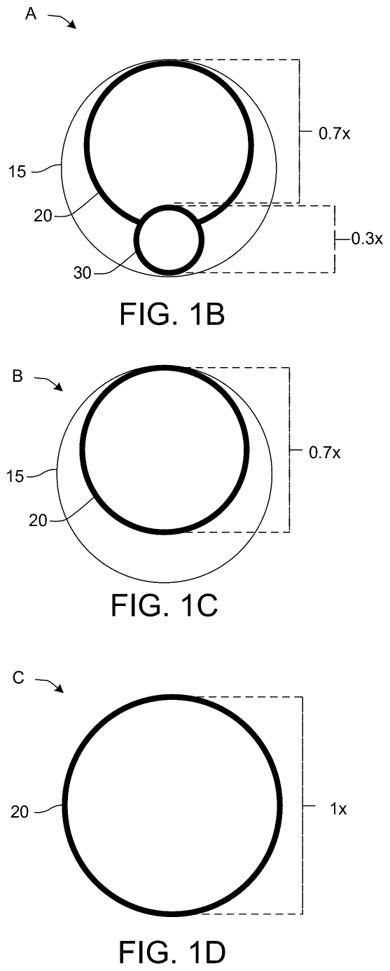

Various embodiments of the present disclosure are described in detail in reference to FIGS. 1A to 3.

Referring to FIG. 1A, a catheter 10 includes a plurality of lumens, such as a first lumen 20 and a second lumen 30. The lumens may be enclosed within a braided sheath 15. Further, the catheter 10 includes one or more transition points, such as a proximal transition point 12 and a distal transition point 14. The transition points of the catheter 10 may be configured to divide the catheter 10 into one or more portions. The stiffness of each portion increases from a proximal end to a distal end of the catheter 10. For example, the proximal transition point 12 and the distal transition point 14 may divide the catheter 10 into a proximal portion, a middle portion, and a distal portion (such as distal portion 23). The stiffness of the proximal portion may be greater than the stiffness of the middle portion, and the stiffness of the middle portion may be greater than the distal portion 23. The length of each of the portions of the catheter 10 may be determined based on requirements. In some embodiments, the braided sheath 15 may be configured along the entire length of the catheter 15. In other embodiments, the braided sheath 15 may enclose the lumens from the proximal end/proximal transition point 12 to the distal transition point 14 of the catheter 10.

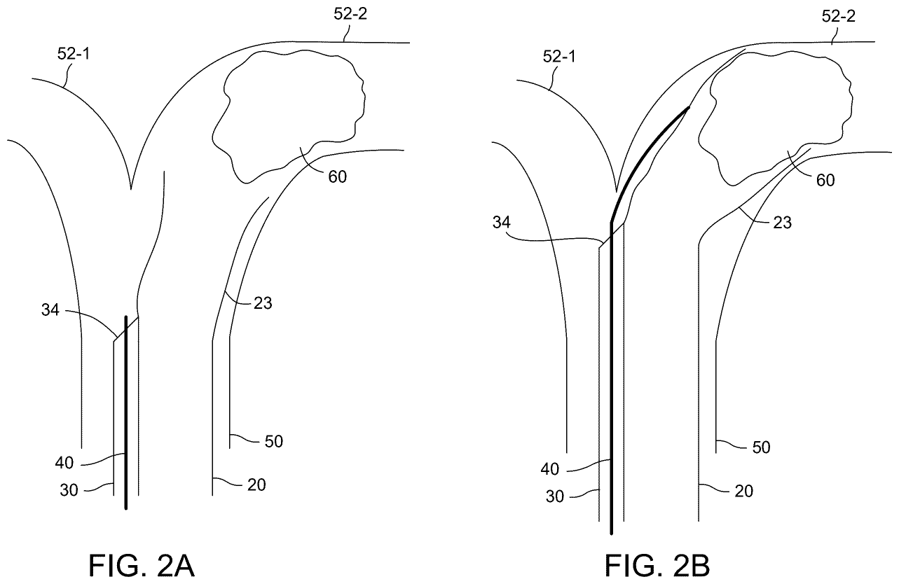

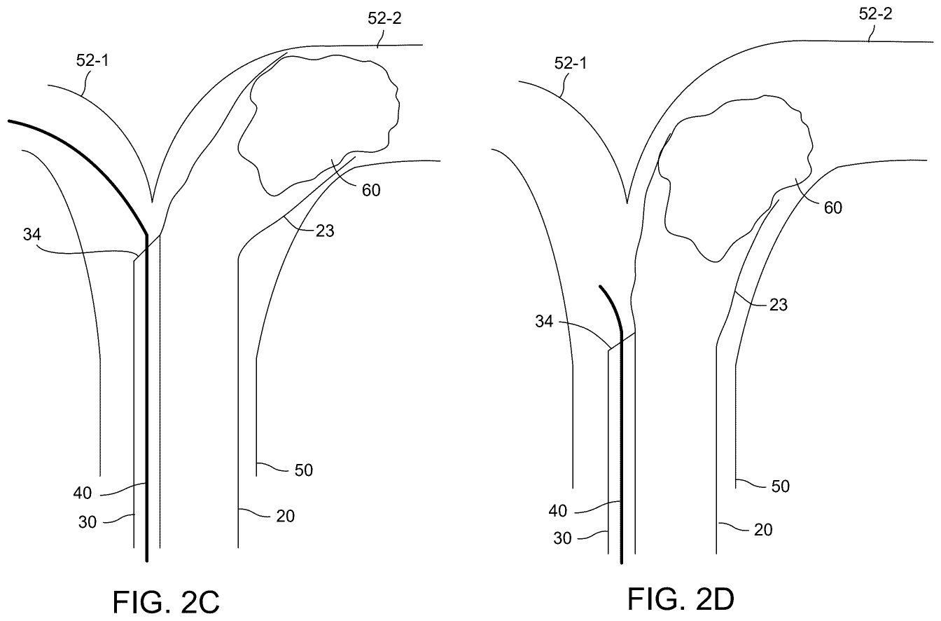

The first lumen 20 may be connected to a suction means (not shown) to allow for aspiration of the. The suction means may be any one or a combination of aspiration syringes, vacuum pumps, electrical suction units, and the like, but not limited thereto. The first lumen 20 may include a first lumen proximal end 22, to which the suction means may be connected. The suction means may be configured to aspirate a blockage or a clot (such as clot 60 shown in FIGS. 2A to 2D) into the first lumen 20. The suction means may be configured to operably generate negative pressure within the first lumen 20 to draw/pull the clot 60 towards the first lumen 20/catheter 10.

The first lumen 20 may also include/form the distal portion 23. The distal portion 23 may extend outward (or in a distal direction) from the distal transition point 14. The distal portion 23 may be substantially flexible, and may be configured to change shape or contour thereof during aspiration. The flexibility of the distal portion 23 may cause it to enclose over the blockage or the clot 60 during aspiration. In some embodiments, the shape/contours of the distal portion 23 may be designed such that the distal portion 23 has a maximal contact area with the blockage or the clot 60 when aspirated. In some embodiments, the distal portion 23 may be configured to flare out from the distal transition point 14, as described subsequently in the present disclosure. In some embodiments, the first lumen 20 may include a distal tip 24 at the distal end of the catheter 10. The first lumen 20 may be configured to receive and enclose the clot 60 through the distal tip 24.

The second lumen 30 may be configured to allow passage of a guiding means (such as guiding means 40 shown in FIGS. 2A to 2D) therethrough. In some embodiments, the guiding means 40 may be at least one of a guide wire, a microcatheter, or a stent retriever. In other embodiments, the guiding means 40 may be any other endovascular device. The guiding means 40 may be inserted into the second lumen 30 through an entry opening 32. The entry opening 32 may be between the proximal end of the catheter 10 and the proximal transition point 12.

In some embodiments, the second lumen 30 may extend up to the distal transition point 14. For example, an orifice 34 (i.e. a distal end of the second lumen 30) may be at or near the distal transition point 14 of the catheter 10. In some embodiments, the orifice 34 may be configured to allow the guiding means 40 (or an end thereof) to exit the second lumen 30. In some embodiments, the orifice 34 may be shaped/placed to allow the guiding means 40 to exit the catheter 10. In such embodiments, the guiding means 40 may allow the guiding means 40 to abut against any surface (such as a blood vessel or a branch vessel) to secure the position of the catheter 10. In some embodiments, the guiding means 40 may be extended either collinearly or obliquely out from the orifice 34, with respect to the catheter 10 (such as in examples shown in FIGS. 2B and 2C, respectively). In other embodiments, the orifice 34 may be placed to allow the guiding means 40 to enter into the first lumen 20. In such embodiments, the guiding means 40 may allow the distal portion 23 of the first lumen 20 towards the blockage/clot 60. After guiding the distal portion 23 to the site of the clot 60, the guiding means 40 may be retracted/withdrawn for unimpeded aspiration through the first lumen 20.

In some embodiments, the entry opening 32 of the second lumen 30 may be defined between the proximal end of the catheter 10 and the distal transition point 14. Such embodiments may allow rapid exchange of the catheters 10. For example, some interventions require a first catheter to be replaced/exchanged with a second catheter, after the first catheter is inserted and the guiding means 40 is made to abut against the surface of the blood vessel. In such examples, the catheters 10 may be exchanged without removing/removing the guiding means 40 from the blood vessel. Reducing the length of the second lumen 30, and having the entry opening 32 defined along the length of the proximal portion, may allow the first catheter to be removed easily while the guiding means 40 remains in position, and the second catheter to be inserted over the guiding means 40. In some embodiments, the first and second catheters may be two of the catheters 10 with the same or different design parameters/dimensions. In other embodiments, the first catheter may be the catheter 10, and the second catheter may be a different type of catheter, or vice versa.

Design parameters such as length, width, materials, and stiffness/flexibility of the catheter 10 across the portions thereof may be determined based on the requirements. For example, the catheters 10 used for removing blockages in the brain may have smaller widths in comparison to those required for removing blockages in the lungs or the heart. Similarly, the length and flaring of the distal portion 23 of the catheter 10 (and corresponding dimensions of the lumens) may be determined based on the location of vascular access (such as based on whether the catheter 10 is inserted through the groin or the wrist of the patient).

The lengths of the catheter 10 may also be adapted based on requirements. In some embodiments, the lengths of the proximal portion, the middle portion, and the distal portion 23 may be about 0.2, 0.3 and 0.5 times the length of the catheter 10, respectively. In other embodiments, the distal portion 23 may have a length between about 0.05 times to about 0.5 times that of the catheter 10. The lengths of each of the portions may be suitably adapted based on the location/site of the clot 60 and the location of the vascular incision, among other requirements.

Referring to FIGS. 1B to 1D, diameters of the first lumen 20 and the second lumen 30 may vary along the lengths thereof. FIGS. 1B, 1C, and 1D show cross-sectional representations of the catheter 10 at lengths/points A, B, and C, respectively, shown in FIG. 1A. In the examples shown, the diameter of the catheter 10 is represented using ‘x’.

In some embodiments, the first lumen 20 may have a diameter between about 0.6 to about 0.8 times that of the catheter 10, at least along the length of one of the portions of the catheter 10. In such embodiments, the second lumen 30 may be configured to have a diameter between about 0.2 to about 0.4 times that of the catheter 10, corresponding to the diameter of the first lumen 20. For example, as shown in FIGS. 1B and 1C corresponding to cross-sectional points A and B of FIG. 1A, the first lumen 20 may have a diameter of about 0.7 times the diameter of the catheter 10, and the second lumen 30 may have a diameter of about 0.3 times the diameter of catheter 10. In some embodiments, the diameter of the first lumen 20 may increase and become equal to the catheter 10 from the distal transition point 14 to the distal tip 24 of the first lumen 20, as shown in FIG. 1D corresponding to cross-sectional point C of FIG. 1A. In such embodiments, the distal portion 23 may gradually or incrementally flare out from the distal transition point 14 to the distal tip 24.

While FIG. 1A shows the catheter 10 having two lumens, it may be appreciated by those skilled in the art that the catheter 10 may be suitably adapted to include any number of lumens, based on the requirements. For example, some medical interventions may require three lumens, such as when different lumens are used for inserting microcatheters for securing the position of the catheter 10, inserting the stent retrievers such that they are caught by the clot 60, and aspirating the clot 60 using the suction means. Further, while the present disclosure describes the use of catheters 10 to remove blockages such as the clot 60, the catheter 10 may be adaptable for removing other kinds of blockages such as including, but not limited to, fat blockages/plaques, cancerous tissues, cysts, inflammations, and the like.

Referring to FIGS. 2A to 2D the catheter 10 may be used for vascular interventions such as embolectomy and/or thrombectomy, among other procedures that require removal of blockages or clots 60. As shown in FIG. 2A, the catheter 10 may be inserted into a blood vessel 50. Vascular access may be provided by making vascular incisions on the patient's body, such as at wrists, or groin. Further, the guiding means 40 (such as a microcatheter), may also be inserted through the second lumen 30. The guiding means 40 may be used to guide/navigate the first lumen 20 through the blood vessel 50.

In some examples, the blood vessel 50 may have one or more branch vessels, such as first and second branch vessels 52-1, 52-2 (collectively referred to as branch vessels 52). The second branch vessel 52-2 may have the clot 60 while the first branch vessel 52-1 may be unaffected, in the examples shown in FIGS. 2A to 2D.

The catheter 10 and the guiding means 40 may be inserted and pushed towards the clot 60, as shown in FIGS. 2B and 2C. The guiding means 40 may be caused to exit the catheter 10 through the orifice 34. In some embodiments, the guiding means 40 may be extended collinearly with respect to the catheter 10/first lumen 20, thereby allowing the guiding means 40 to push and guide the first lumen 20 (or at least the distal portion 23 thereof) towards the clot 60, as shown in FIG. 2B. Once the first lumen 20 is proximate to the location of the clot 60 such that the distal tip 24 encloses the clot 60, the guiding means 40 may be withdrawn, such as to a position shown in FIG. 2A. The contours of the distal portion 23 and the distal tip 24 may be suitably adapted to allow the clot 60 to be enclosed with the distal portion 23 and the distal tip 24. In some embodiments, the orifice 34 may be configured to cause the guiding means 40 to exit the second lumen 30 and enter into the first lumen 20. In such embodiments, the guiding means 40 may be configured to guide the first lumen 20 from within.

In other embodiments, the orifice 34 may cause the guiding means 40 to exit the catheter 10, and guide the first lumen 20 externally, as shown in FIG. 2B. The guiding means 40 may also be extended obliquely from the second lumen 30, with respect to the catheter 10/first lumen 20. Further, in such embodiments, the guiding means 40 may be pushed towards the branch vessel 52-1, as shown in FIG. 2C. The guiding means 40 may be configured abut against an inner wall of the blood vessel 50 or the branch vessel 52-1 (or generally any surface) to anchor and secure the position of the catheter 10.

The suction means may then be configured to aspirate the clot 60 into the first lumen 20. The aspiration may cause the clot 60 to be pulled/drawn into the distal portion 23, and the distal tip 24 and the distal portion 23 may be caused to close over the clot 60. In some embodiments, since the distal portion 23 is flexible and flares out, the distal portion 23 may have a greater contact area/surface area with the clot 60 when the distal portion 23 closes over/encloses the clot 60, in comparison to existing catheters. Further, since the guiding means 40 are inserted through the second lumen 30, the catheter 10 may allow for unimpeded aspiration through the first lumen 20, which increases suction power and reliability of suction.

Once the clot 60 is aspirated and securely enclosed within/adhered to the distal portion 23, the guiding means 40 and the catheter 10 may be withdrawn from the blood vessel 50, as shown in FIG. 2D. Since the clot 60 is enclosed within the distal portion 23, the clot 60 may be pulled out along with the catheter 10.

In other examples, the blood vessel 50 may not have branch vessels 52 near/proximate to the clot 60. In such examples, the guiding means 40 may guide the first lumen 20 linearly through the blood vessel 50, and position the first lumen 20 such that the distal portion 23 encloses over at least a part of the clot 60/blockage. The guiding means 40 may be configured caused to abut against the inner wall of the blood vessel 50, or withdrawn from the catheter 10. The first lumen 20 may be used for aspirating the clot 60, and the catheter 10 may be withdrawn from the blood vessel 50 thereafter to remove the clot 60/blockage.

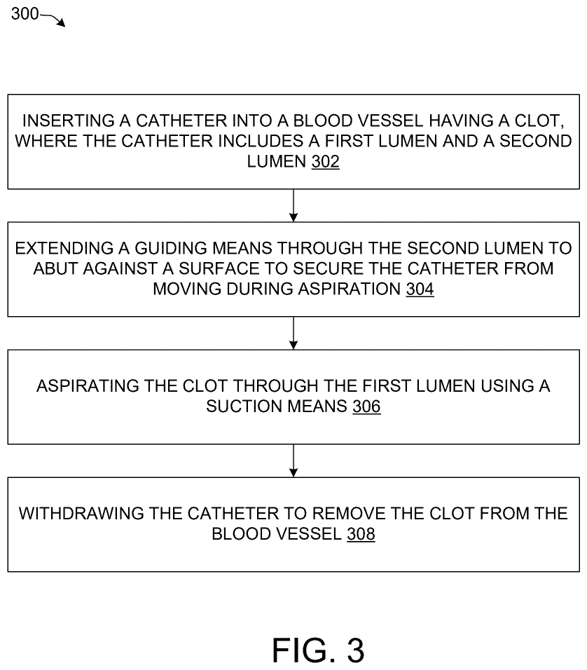

Referring to FIG. 3, an example method 300 for vascular interventions using catheters with multiple lumens includes the steps 302 to 308.

At step 302, the method 300 includes inserting a catheter into a blood vessel having a clot (such as the blood vessel 50 and the clot 60 of FIGS. 2A to 2D). The catheter may be the catheter 10 of FIGS. 1A to 2D, having at least the first lumen 20 and the second lumen 30.

At step 304, the method 300 includes extending a guiding means (such as the guiding means 40) through the second lumen 30 to abut against a surface to secure the catheter 10 from moving during aspiration. In some embodiments, the surface may be at least one of an inner wall of a blood vessel (such as blood vessel 50), or a branch vessel (such as branch vessels 52-1, 52-2). In some embodiments, the guiding means 40 may be configured to guide/navigate the catheter 10 through the blood vessel 50 towards the clot 60.

At step 306, the method 300 includes aspirating the clot 60 through the first lumen using a suction means, such as suction means described previously in the present disclosure. In some embodiments, the distal tip 24 or the distal portion 23 of the first lumen 20 may enclose over and adhere to at least a portion of the clot 60 during aspiration.

At step 308, the method 300 includes withdrawing the catheter 10 to remove the clot 60 from the blood vessel 50.

The present disclosure solves the problems associated with single lumen catheters, viz. the problem of guiding means 40 pushing or breaking the clot 60, and impeding the suction of force during aspiration. The catheter 10 of the present disclosure solves at least the aforementioned problems by including multiple lumens having at least the first lumen 20 for aspiration, and the second lumen 30 for insertion and withdrawal of the guiding means 40. By using the second lumen 30 for the guiding means 40 and the first lumen 20 for aspiration, the second lumen 30 may prevent the guiding means 40 from impeding the aspiration/suction by the suction means. The catheter 10 may allow for stronger and more reliable aspiration during vascular interventions.

Further, the catheter 10 of the present disclosure allows the guiding means 40 to abut against and anchor to different branches of blood vessels while the first lumen 20 is directed towards the clot 60 for aspiration. In such implementations, the guiding means 40 may better secure the position of the catheter 10, thereby further improving strength and reliability of aspiration. Additionally, the flexibility and the flaring out of the distal portion 23 of the first lumen 20 may improve contact area/surface area with the clot 60 during aspiration, which is desirable for safe and reliable removal of the clots 60.

Although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above-described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. The skilled person will be aware of a range of possible modifications of the various embodiments described above. Accordingly, the present disclosure is defined by the claims and their equivalents.

What is claimed is:

1. A catheter, comprising:
a plurality of lumens having at least:
a first lumen connected to a suction means for aspiration; and
a second lumen that allows passage of a guiding means, wherein the second lumen extends up to a distal transition point of the catheter.

2. The catheter of claim 1, wherein the second lumen comprises an orifice configured to allow the guiding means to exit the catheter.

3. The catheter of claim 2, wherein the guiding means is configured to abut against at least one of: an inner wall of a blood vessel, or a branch vessel, for securing position of the catheter.

4. The catheter of claim 2, wherein the guiding means is extendible either collinearly or obliquely out from the orifice with respect to the catheter.

5. The catheter of claim 1, wherein the guiding means is at least one of: a guide wire, a microcatheter, or a stent retriever.

6. The catheter of claim 1, wherein the plurality of lumens are enclosed in a braided sheath of the catheter.

7. The catheter of claim 1, wherein the first lumen comprises a distal portion that is flexible.

8. The catheter of claim 7, wherein the distal portion is configured to enclose over a clot during aspiration.

9. The catheter of claim 1, further comprising one or more transition points that divide the catheter into one or more portions, and wherein stiffness of each portion increases from a proximal end to a distal end of the catheter.

10. The catheter of claim 9, wherein the one or more transition points comprises a proximal transition point and the distal transition point that divide the catheter into a proximal portion, a middle portion, and a distal portion.

11. The catheter of claim 10, wherein the proximal portion, the middle portion, and the distal portion are about 0.2, 0.3 and 0.5 times the length of the catheter, respectively.

12. The catheter of claim 10, wherein the second lumen comprises an entry opening located between a proximal end of the catheter and the distal transition point.

13. The catheter of claim 7, wherein the distal portion has a length between 0.05 times to 0.5 times that of the catheter.

14. The catheter of claim 1, wherein the first lumen has a diameter between 0.6 to 0.8 times that of the catheter.

15. The catheter of claim 1, wherein the diameter of the first lumen increases from the distal transition point to a distal tip of the first lumen.

16. The catheter of claim 1, wherein the second lumen has a diameter between 0.2 to 0.4 times that of the catheter.

17. A method for vascular interventions, comprising:
inserting a catheter into a blood vessel having a clot, wherein the catheter comprises a first lumen and a second lumen;
extending a guiding means through the second lumen to abut against a surface to secure the catheter from moving during aspiration;
aspirating the clot through the first lumen using a suction means; and
withdrawing the catheter to remove the clot from the blood vessel.

18. The method of claim 17, wherein for inserting the catheter the method comprises guiding the catheter towards the clot using the guiding means.

19. The method of claim 17, wherein a distal tip of the first lumen encloses over at least a portion of the clot during aspiration.

20. The method of claim 17, wherein the surface is at least one of: an inner wall of a blood vessel, or a branch vessel.
